(19) United States
(12) Patent Application Publication
(10) Pub. No.: US 2005/0059103 A1
(43) Pub. Date: Mar. 17, 2005

(54) Means and methods for the detection of immunoglobulin capable of binding to mycobacterium antigen

(76) Inventors: Jacky Theo Maria Veuskens, Hasselt (BE); Robertus Petrus Maria Van Gijlswijk, Alphen aan den Rijn (NL); Maria Louise Elizabeth Peekel, Heemskerk (NL)

Correspondence Address: Ronald J Baron;Hoffman & Baron, 6900 Jericho Turnpike, Syosset, NY, 11791, US

(21) Appl. No.: 10/495,040

(22) PCT Filed: Nov. 8, 2002
(86) PCT No.: PCT/NL02/00717

(30) Foreign Application Priority Data
Nov. 9, 2001 (EP) 01204285.9

(51) Int. Cl. G01N 33/554; G01N 33/569

(52) U.S. Cl. 435/7.32

(57) ABSTRACT

The invention provides a method for determining whether an animal has been exposed to a mycobacterium or immunological equivalent thereof comprising providing a collection of mycobacterium antigens and determining whether a sample from said animal comprises an immunoglobulin capable of binding to at least one of said mycobacterium antigens, the method characterized in that said at least one mycobacterium antigen is bound to a solid phase through a linking moiety. Said antigen is bound to a solid phase through a linking moiety to obtain a sensitive and specific diagnostic test for mycobacterium. Preferably, said antigen comprises a label. Said label can easily demonstrate binding of said immunoglobulin to said antigen. In one aspect, the invention provides a kit for performing a method of the invention, comprising a collection of mycobacterium antigens, wherein at least one of said antigens is bound to a solid phase through a linking moiety.

The invention relates to the field of medicine. More in particular the invention relates to the field of diagnostics.

The genus Mycobacterium contains about 50 species. It is responsible for a number of diseases which are known collectively as mycobacterioses. The best known and widest spread of these are leprosy, caused by M. leprae, and tuberculosis caused by M. tuberculosis. Both of these diseases affect more than ten million people all over the world. Most other mycobacteria normally occur only as environmental saprophytes. However, they can also cause opportunist diseases, which happens often, but not exclusively, in organisms suffering from problems with their immune systems, such as AIDS patients or people undergoing immunosuppression. The opportunist types comprise the slow-growing species M. avium, and the closely related M. intracellulare and M. scrofulaceum (often together referred to as the MAIS complex), M. kansai, M. marinum and M. ulcerans, and the fast-growing species M. chelonae and M. fortuitum. Although quite rare in the Western world for several decades, the occurrence of opportunist mycobacterial diseases and tuberculosis has shown a significant increase with the incidence of AIDS. Further, it has been reported that mycobacteria or antigens of mycobacteria play a role in the etiology of a plurality of other diseases, such as sarcoidosis and Crohn's disease, as well as different auto-immune diseases, such as auto-immune dermatitis, rheumatoid arthritis and auto-immune diabetes mellitus. It has been suggested that this role can be attributed to a structural mimicry between epitopes of mycobacteria and those of the host organism.

The cell walls of mycobacteria are very complex and contain many different lipids, some of which have structures unique to the genus. These structures comprise, amongst others, mycolinic acids and esters, peptido-glycolipids, arabino-galactane and lipo-arabinomannane. The lipid-rich cell walls of a mycobacterial cell are responsible for the notable ‘acid fast’ coloring properties of the mycobacteria. They also enable mycobacteria to counterman attack by the immune system of a host organism. A number of species, after being taken up into macrophages, surround themselves with a thick layer of secreted lipids.

Many of the different components of the mycobacteria interact with the immune system of a host organism. These components comprise mainly proteins and hydrocarbon antigens, which can either be actively secreted by the mycobacteria or can form part of the cell wall or cell membrane. In addition, they may be present in the cytoplasm, for example in the cytoplasmic matrix, ribosomes and enzymes. Mycobacteria further also possess immuno-modulating components, such as immunosuppressing compounds and adjuvants. As of consequence, a single mycobacterial species can induce a large variety of immune responses in different forms having diverse specificities. This makes it very difficult to derive antigens which are suitable for the detection of species-specific humoral responses as a basis for a highly sensitive and specific diagnostic test for the above mentioned diseases, particularly tuberculosis. Because saprophytic mycobacteria occur ubiquitously, both human and animal body fluids contain nearly at all times anti-mycobacterial antibodies.

Many researchers have attempted to develop sufficiently specific and sensitive diagnostic tests for mycobacterioses. For example in tuberculosis diagnostics the basic idea was, with the aid of recombinant technology, to search for unique bio-organic molecules. To date the identification, purification, and characterisation of several specific recombinant antigens has been described. Despite their specific nature none of these display sufficient sensitivity in a diagnostic test for the detection of specific humoral immune responses. Improving the quality of an antigen can be achieved through the use of a recombinant antigen raised in the specific host itself whenever possible. However, the use of non-recombinant antigens is preferred, because such antigens are for a large part processed as occurs in nature. To further improve the overall performance of the test it was shown that more than one antigen should be used for the immunological detection of tuberculosis. Mixing of antigens at predefined ratio's improves the sensitivity of the diagnostic test without decreasing the specificity.

A method of presenting data of clinical (test) trials is the so-called Receiver Operator Curve (ROC curve). A ROC curve presents the relation between sensitivity and specificity for the respective tests in the art. The curve depicts both the optimal sensitivity versus specificity and the robustness of the test around this optimum. In addition to sensitivity and specificity, a third parameter of a diagnostic test is the area under the curve (AUC) in the ROC curve. AUC is a measure for the discrimination power of the test between a group of patients and a group of reference subject (e.g. healthy individuals). The value of AUC equals the probability that a randomly chosen reference subject has an OD ratio smaller than or equal to the OD ratio for a randomly chosen patient.

In spite of the above-mentioned approaches of development of diagnostic tests for mycobacterioses, a problem encountered with current diagnostic tests is the poor sensitivity versus specificity results that are still obtained. Until the present invention, no reliable diagnostic test was available for mycobacterioses with sufficient sensitivity and specificity.

The present invention provides tests with improved ROC-curves. The invention further provides means and methods for generating and using such tests. An important factor of a method of the invention for improving the sensitivity and specificity of a diagnostic test for mycobacterioses is the quality of antigen(s) provided. Another important factor of a method of the invention for improving the sensitivity and specificity of a diagnostic test for mycobacterioses is the choice of combination of antigen(s) to be included in the test. Mycobacteria produce many different kinds of molecules that can elicit immune responses in a host and induce the production of antibodies. The present invention provides methods for selecting collections of antigens which, when used in a diagnostic method of the invention, produce improved ROC-curves.

It was observed that association of antigen with a solid phase via a linking moiety results in improved sensitivity and specificity of diagnostic tests for mycobacterioses. Surprisingly, said sensitivity and specificity id improved in such extent, that reliable diagnostic tests for mycobacterioses have now become possible. The cause of this major improvement of the test performance through the use of a linking moiety is still unclear. Without being bound by theory, a possible explanation of said improvement of a mycobacterium test performance through the use of a linking moiety is that direct binding of an antigen to a solid phase induces slight structural (e.g. conformation and/or configuration) alterations in mycobacterium antigen. Also, a linker places an antigen further away from the surface of a solid support possibly improving the accessibility of said antigen and/or interaction between antibody and antigen. It was found that conventional diagnostic tests for mycobacterioses, which do not involve binding of an antigen to a solid phase through a lining moiety, already result in sub optimal ROC-curves. Probably, even slight alterations in the structure or the accessibility of a mycobacterium antigen as a result of direct binding of said antigen to an ELISA dish already alters the performance of a mycobacterium diagnostic test to such an extent that the ROC-curve is negatively influenced. Until the present invention, it was not known that the use of a linking moiety can change a non-significant diagnostic test into a significant and reliable test.

Directly bound mycobacterium antigens to a solid phase are not suitable for a sensitive and specific mycobacterium diagnostic test. This poses a problem since most tests, at one point or another, require association of antigen with a solid phase, if only to wash away unbound immunoglobulin. In the present invention it has been found that direct association of mycobacterium antigen with a solid phase influences the ROC-curve negatively. For this reason it is preferred that said antigen is associated with a solid phase through a linking moiety.

The invention thus provides a method for determining whether an animal has been exposed to a mycobacterium or immunological equivalent thereof comprising providing a collection of mycobacterium antigens and determining whether a sample from said animal comprises an immunoglobulin capable of binding to at least one of said mycobacterium antigens, the method characterized in that binding of said antigen to a solid phase is achieved through a linking moiety. Preferably, said linking moiety mediates binding of all antigens to a solid phase. After said antigen(s) are provided with a linking moiety, they can be exposed to a sample from an animal. If said sample comprises antibodies against said mycobacterium antigens, said antibodies are now sufficiently capable of binding said antigen. Bound antibodies can subsequently be visualised by common immunological methods in the art, for instance by staining with labelled antibodies against human IgG. With a method of the invention, a sufficiently sensitive and specific diagnostic test for mycobacterioses has now been provided. Until the present invention it was unexpected that a collection comprising different antigens would all uniformly exhibit improved results in a diagnostic test because of one general action performed on them. Because of their different properties, different antigens are more likely to behave differently if they are all treated in one specific manner. In the present invention, however, it is shown that the use of a linking moiety renders a diagnostic test reliable for each antigen at once.

Of course, it is clear for a person with ordinary skills in the art that the order of steps of a method of the invention can be reversed. For instance, mycobacterium-specific antibodies can be linked to a solid phase, after which said antibodies can be exposed to mycobacterium antigens. Binding of said antigens can be visualised using common techniques known in the art. A method of the invention wherein the order of steps is reversed, lies as well within the scope of the invention and leads to improved sensitivity and specificity compared to methods that do not use a linking moiety to bind a mycobacterium antigen bound to a solid phase. In one embodiment of the invention, mycobacterium-specific antibodies linked to a solid phase age provided with a sample from an animal. If said sample comprises mycobacterium antigens, said antigens are now sufficiently capable of binding said antibodies. The invention therefore also provides a method for determining whether an animal has been exposed to a mycobacterium or immunological equivalent thereof comprising providing mycobacterium-specific antibodies and determining whether a sample from said animal comprises a mycobacterium antigen. However, although a method of the invention can be used to determine the presence of mycobacterium antigens in a sample, a method of the invention is preferably used for determining whether a sample comprises one or more immunoglobulins capable of binding one or more mycobacterium antigens. Detection of mycobacterium-specific immunoglobulins is preferred because this is a more sensitive and more specific test.

In another embodiment, mycobacterium antigen-antibody complexes are formed prior to being bound to a solid phase. After said antigen-antibody complexes are formed, said complexes are linked to a solid phase.

Prevention of direct association of antigen with a solid phase is not only important in a detection or typing assay of the invention. It is also important in purification protocols. In order to provide for antigen that can be used in a test of the invention, mycobacterium antigens can be purified from a suitable source. Several purification methods are available in the art. Depending on the separation step used, several fractions are obtained of which typically some are pooled to provide a collection of antigens used in a test of the invention. Strikingly, separation procedures that do not require antigen for the assay to be directly bound to a solid phase result in better antigen preparations than separation procedures that do require the direct binding of antigen to be used in a test of the invention, to a solid phase. Thus in a preferred embodiment a method as depicted above further comprises providing antigen that has not been directly associated with a solid phase during its production and/or purification. Preferably, said purification comprises gelfiltration.

An immunological equivalent of a mycobacterium can elicit the same humoral immune response effect in kind, not necessarily in amount, as an immunogen from said mycobacterium. Currently, immunological equivalents are preferred for vaccination purposes, because they involve less risk of uncontrolled infection events than the natural pathogen from which they are derived. As a method of the invention is suitable for determining whether an animal has been exposed to an immunological equivalent of a mycobacterium, a method of the invention can thus also be used to determine whether an animal has received a vaccine for said mycobacterium. A method of the invention can also be used to determine the effect of an immunization of said animal.

A mycobacterium antigen is defined herein as a molecule that is derived or in essence derivable from said mycobacterium, wherein said molecule is capable of eliciting or boosting a humoral immune response against mycobacterium in an animal, either alone or in combination with a suitable adjuvant.

By “an animal has been exposed to a mycobacterium or immunological equivalent thereof” is meant that said mycobacterium or immunological fragment thereof has been present in and/or on said animal. Said mycobacterium or immunological fragment thereof may have been present in said animal as a result of a natural infection. A natural infection involves entering of a (spore of a) mycobacterium in and/or on said animal and subsequent multiplying of said mycobacterium. Alternatively, said mycobacterium or immunological equivalent thereof may have been provided to said animal artificially. For instance, said animal may have been vaccinated. Said mycobacterium or immunological equivalent thereof does not necessarily have to be present in and/or on said animal at the time that said animal is tested for said mycobacterium or immunological equivalent thereof. As used herein the term animal is also used to refer to a human.

A linking moiety can be any molecule that binds to said antigen with sufficient strength and/or affinity as to warrant binding under normal washing conditions for immunology based diagnostic tests, but that preferably at the same time does not provide a target for animal immunoglobulin and preferably does not induce immunologically significant structural changes in said antigen. A linking moiety must also be able to bind to a solid phase either directly or indirectly. Direct binding can be through covalent or non-covalent interaction with the solid phase. A linking moiety can also be indirectly linked to a solid phase. This indirect linkage can be through any means. Suitable linking moieties are for instance immunoglobulins or functional equivalents thereof. A functional equivalent of such an antibody comprises the same antigen binding activity in kind, not necessarily in amount. Suitable equivalents are for instance FAB-fragments, single chain antibodies, synthetically or naturally produced antibodies, artificially generated antibodies, protein A, protein G, avidin, streptavidin, etc.

In a preferred embodiment at least one of said antigens comprises a label. More preferably, all of said antigens comprise a label. Using a label it is possible to utilize standardized and optimised embodiments of a diagnostic method of the invention. Binding of an immunoglobulin to said labelled antigen can easily be demonstrated. For instance, in a capture ELISA immunoglobulin bound to a solid phase can be provided with labelled antigen. After incubation, unbound labelled antigen is washed away. After said washing, the presence of said label clearly indicates the presence of bound antigen and, hence, the presence of an immunoglobulin capable of binding said antigen. Labelled antigens are as well suitable for immunocomplex ELISA and indirect coating ELISA, as is shown in the examples. Attaching a label to said antigen is preferably achieved using methods that do not significantly alter the configuration of said antigen. Any type of label that is able to present a target for a binding molecule is in principle suitable. Preferably, said label comprises a label for which a suitable capture reagent exists. In a preferred embodiment said label comprises fluorescein, dinitrophenol, biotin, digoxin, and/or digoxigenin.

In one embodiment of the invention mycobacterial specific immunoglobulin comprises a label. In this particular embodiment said collection of mycobacterial antigen may be linked to a solid surface directly prior to detection thereof through binding of said labelled immunoglobulin. Better results with respect to sensitivity and specificity are obtained when also in this embodiment at least one mycobacterial antigen is linked to said solid surface using a method of the invention i.e. through a linking moiety.

In a preferred embodiment a linking moiety of the invention comprises an immunoglobulin, or a functional equivalent thereof, specific for immunoglobulin of said animal. Tests comprising this preferred embodiment exhibit improved sensitivity and specificity. More preferably, said linking moiety comprises an immunoglobulin, or a functional equivalent thereof, specific for said (labelled) antigen(s). Said immunoglobulin is particularly well capable of binding said antigen(s). Diagnostic tests comprising this preferred embodiment exhibit improved sensitivity and specificity. In an even more preferred embodiment (labelled) antigen(s) are bound to immunoglobulin, or a functional equivalent thereof, prior to binding of said antigens to a solid phase. Also this embodiment leads to tests with improved ROC-curves. In a particularly preferred embodiment an immunoglobulin specific for labelled antigen is specific for the label part of said labelled antigen. In this way many different antigens can be bound using a single immunoglobulin.

In a particularly preferred embodiment of a method of the invention, said linking moiety comprises an immunoglobulin, or a functional equivalent thereof, specific for the label of labelled antigens, and said labelled antigens are bound to immunoglobulin, or a functional equivalent thereof, prior to binding of said antigens to a solid phase. Preferably, said immunoglobulin bound to said antigen prior to binding of said antigen to said linking moiety, comprises an immunoglobulin derived from said animal.

A solid phase can be a solid surface. A (magnetic) bead is also a solid phase. Porous material also has solid phases making up the wails of the pores. Superficial association with such a wall during passage of fluid containing said antigen, for instance due to random bouncing, is not considered association with said solid phase. Typically associations that are so weak as not to require a change in environment for the association to break are not considered associations in the present invention. A change in environment in this respect refers to, for instance, a minor change in temperature, pH, salt concentration or solvent. Of course other changes in environment can also be envisaged.

It has been observed in the past that diagnostic assays, for determining whether an animal has been exposed to mycobacteria, improve in discriminating power when they comprise more than one mycobacterium antigen. Therefore a method of the invention preferably comprises a collection comprising at least two different mycobacterium antigens. More preferably, said collection comprises between about five and fifteen different mycobacterium antigens. Said different antigens are obtainable in several ways, for instance by pooling of fractions obtained with a chromatographic separation method. Preferably, said chromatographic separation method comprises a method wherein direct binding of antigen to solid phase is avoided. Preferably said chromatographic separation method comprises gelfiltration. With gelfiltration direct binding of antigens to a solid phase is avoided. This leads to better results compared to a method wherein antigens are directly bound to a solid phase. Fractions obtained with a chromatographic separation method preferably comprise about 3-4 antigens. In a more preferred embodiment, said collection comprises fractions A, B and/or C from the RISA 200 antigen mixture as described in the examples. Even more preferably, said collection comprises antigens from all fractions obtainable by a method as described in the examples.

Preferably said antigens are obtained using methods that do not rely on direct interaction of antigen with a solid phase. In this respect, it is preferred that said antigens are derived from culture supernatant of cells comprising said mycobacterium and not from sources that contain whole cells or fractions thereof. Supernatant, as opposed to sources that contain whole cells or fractions thereof, can be obtained that is relatively free of other antigens thus obviating the need for very elaborate purification steps. Preferably, said antigen is obtained using methods that comprise gelfiltration. Tests comprising antigens that were obtained using methods that do not rely on direct interaction with a solid phase exhibit improved sensitivity and specificity. Particularly antigens obtained by gelfiltration are observed to improve sensitivity and specificity of tests involving said antigens, as compared to tests involving antigen obtained using hydrophobic interaction columns.

Mycobacteria of different species have many antigens in common. I.e. binding molecules capable of interacting with antigen obtained from one mycobacterium species are often also capable of interacting with an antigen of another mycobacterium species. This feature can be used in a test of the invention. For instance, a test optimised for use in a human population can also be used in a life stock setting, for instance in cattle. However, typically the affinity of an immunoglobulin for an antigen varies with the mycobacterium species said antigen is derived from. Therefore, preferably at least one of said antigens is derived from a mycobacterium species that said animal is being tested for. More preferably, all said antigens are derived from a mycobacterium species that said animal is being tested for. Preferably, said animal is a mammal, more preferably a human.

As is shown in the examples, a method of the present invention can be well performed by using an indirect ELISA, a capture ELISA and/or an immunocomplex ELISA. In each of said ELISA techniques, an antigen is bound to a solid phase through a linking moiety, resulting in improved sensitivity and specificity. Therefore one embodiment of the invention provides a method of the invention, wherein said binding of said antigen to a solid phase is achieved through an indirect ELISA, a capture ELISA and/or an immunocomplex ELISA.

In another aspect, the invention provides a kit for performing a method of the invention, said kit comprising a collection of mycobacterium antigens, wherein at least one of said antigens is bound to a solid phase through a linking moiety. Preferably, all of said antigens are bound to a solid phase through a linking moiety. In yet another aspect, the invention provides a kit for performing a method of the invention, said kit comprising a collection of mycobacterium antigens, wherein at least one of said antigens are obtained by a method which avoids direct association of said antigen to a solid phase. Preferably, said method comprises gelfiltration.

Linking of a label to an antigen or immunoglobulin is preferably direct, i.e. not through a linking moiety. Thus by covalently linking the label to the antigen or the immunoglobulin.

The invention further provides a collection of mycobacterium antigens comprising at least one mycobacterium antigen that is physically linked to a label. Preferably, said collection comprises a plurality of mycobacterium antigens physically linked to a label. As discussed herein above, the antigen is better suited for a diagnostic assay when it has been collected from a culture supernatant and/or not been associated with a solid surface during its preparation. Thus preferably said antigens are derived from supernatant from an in vitro culture of mycobacteria and/or said antigens have passed through a gelfiltration column. In a preferred embodiment said label comprises fluorescein, dinitrophenol, biotin, digoxin, and/or digoxigenin.

In another embodiment the invention provides a collection of antibodies comprising at least one antibody specific for a mycobacterium antigen physically linked to a label. Preferably said collection is derived from the serum of an animal, preferably a human. Preferably said label comprises fluorescein, dinitrophenol, biotin, digoxin, and/or digoxigenin.

A preferred content of some diagnostic kits according to the present invention is shown below.

Indirect and/or ICx kit

Anti-fluorescein coated solid
support (micro-titre plate, latex,
nitrocellulose, etc)
Dilution and washing buffers
Fluorescein labelled
mycobacterium antigens (e.g.
RISA 200 A/B/C/ - 1/1/1)
HRP anti-human IgG conjugate
HRP substrate (e.g. TMB) and
dilution buffer
Positive control and cut-off sera
Manual and troubleshooting guide

Capture kit

Anti-human Ig coated solid
support (micro-titre plate, latex,
nitrocellulose, etc)
Dilution and washing buffers
Fluorescein labelled
mycobacterium antigens (e.g.
RISA 200 A/B/C/ - 1/1/1)
HRP anti-fluorescein conjugate
HRP substrate (e.g. TMB) and
dilution buffer
Positive control and cut-off sera
Manual and troubleshooting guide

The terms immunoglobulin and antibody are used interchangeably. An immunoglobulin is used herein to refer to binding molecules such as antibodies of the various subtypes, preferably IgG1 or IgG2. However, the term immunoglobulin is also used to encompasses antigen specific binding fragments thereof such as but not limited to FAB fragments. Of course derivatives or synthetic versions comprising essentially the same binding specificity in kind not necessarily in amount are also within the scope of the invention. Synthetic versions ay include versions such as but not limited to single chain Fv fragments. Current technology allows for the selection and use of the antigen binding part of immunoglobulins without the intermediary of an animal or human. Such synthetic variants are also within the scope of the invention.

The invention is further illustrated in the following examples. These examples serve to exemplify the invention and do not limit the scope of the invention in any way.

### EXAMPLES

Material and Methods

Isolation of Mycobacterium Tuberculosis (MTB) Antigens

Culturing of MTB Cells and Subsequent Harvesting of Cells and Culture Supernatant

1. MTB “stam 1” (RIVM 4514.) (Dutch Royal Tropical Institute) is cultured in 15 litre batches for 3 to 6 weeks in Sauton H plus culture medium containing 50 mM glucose, 4 mM Mn, 4 mM Co and 2 mM Zn.
2. Cells are harvested by centrifugation for 30 min. 10.500 rpm (18,644 g). The clear supernatant is passed through a 0.2 μm filter.
   Preparation of Triton-Extract of MTB-Cells (KT3, CT+)
1. Cells (75 g wet weight) are resuspended in 100 ml. 0.5% Triton-X100, 10 mM TrisHCl pH 7.2 and 0.02% NaN₃. Cells are killed by incubating the mixture 1 hr at 55° C. Next, the cells were sonicated twice for 15 minutes in a Branson sonicator at 0° C. Cells are pelleted by centrifugation for 1 hr at 20.000 rpm (35,500 g).
2. The supernatant is collected (Sup. 1) and the remaining pellet is washed with 100 ml 0.5% Triton-X100, 10 mM TrisHCl pH 7.2 and 0.02% NaN₃. Cells are pelleted by centrifugation for 1 hr at 20.000 rpm (35,500 g).
3. The supernatant is collected (Sup. 2) and the remaining pellet is again washed with 100 ml 0.5% Triton-X100, 10 mM TrisHCl pH 7.2 and 0.02% NaN₃. Cells are pelleted by centrifugation for 1 hr at 20.000 rpm (35,500 g).
4. The supernatant is collected (Sup. 3) and sup. 1 to 3 (volume 250 ml) are pooled and cleared by centrifugation 1 hour at 30.000 rpm (53,300 g). This antigen mixture is called CT+.
5. KT3 is prepared by removing the triton using an Extract-Gel D column (Pierce, 20346) according to the instructions of the manufacturer.
   Isolation of MTB-Culture Supernatant Proteins
1. Culture supernatant (CS, volume 13-13.5 l); containing approx. 0.8 mg/ml protein and 0.5 mg/ml carbohydrate, pH7.2-7.4).
2. To 13 l CS 3,703 kg ammonium sulphate (AS) is added (CS is now 45% saturated AS). The pH is adjusted to 4 by adding approximately 70 ml half concentrated HCl (18%). The CS is stirred for 1 hour.
3. Next, the solution is centrifuged for 30 min. at 10.500 rpm (18,644 g).
4. The pellet is solubilized in 150 ml 10 mM sodium phosphate pH 10.0. The pH of this solution is brought to 7 by adding approximately 20 ml of 1 M NaOH (CS-AS, volume approximately 170 ml; containing 12 mg/ml protein and 1.7 mg/ml carbohydrate).
5. Stored at 80° C.
   Preparation of HIC-Purified Culture Supernatant (KS98)
1. CS-AS was desalted using a 5 ml high-trap desalting column (AP-Biotech, 17-1408-01): 0.1.5 ml was applied and eluted in 2 ml PBS.
2. To 10 ml desalted CS-AS 0.33 mg AS was added and loaded onto a 10×150 mm phenylsepharose column (AP-Biotech, 17-1351-0.1) with a flow of 1 m/min.
3. The flow was increased to 2 ml/min and a linear gradient from 0 to 100% B was applied (buffer A: 0.5 M AS in 10 mM Na-phosphate pH8.0; buffer B: 10 mM Na-phosphate pH8.0).
4. Five fractions eluting from 10 to 30% B are collected and combined. This antigen mixture is called KS98.
   Preparation of Superdex75 Gel-Filtration-Purified Culture Supernatant (RISA75 Antigens)
1. One ml of clear CS-AS (approximantly 5 mg protein) is loaded onto a 8×300 mm Superdex75 HR 10/30 column (AP-Biotech, 17-1047-01).
2. The flow was 0.6 ml/min in PBSE (PBS/2 mM EDTA). Fractions of 0.6 ml were collected. Only fractions eluting at 8.0-9.2 ml (fraction A); 9.2-11.0 ml fraction B) and 11.0-12.8 ml (faction C) are collected. Fractions eluting at other volumes are stored at −20° C. until further use.
3. Measure protein concentrations using BCA reagent (Pierce, 23220) according to the instruction of the manufacturer.
4. Mix fractions A, B and C in a 1:2:2 ratio (w/w). This antigen mixture is called RISA75. To obtain a good mixing ratio, different amounts of fractions A, B and C were mixed, labelled and tested for different ELISA format types.
   Preparation of Superdex200 Gel-Filtration-Purified Culture Supernatant (RISA200 Antigens)
1. One ml of clear CS-AS (approximately 5 mg protein) is loaded onto a 8×300 mm Superdex200 HR 10/30 (AP-Biotech, 17-1088-01) column.
2. The flow was 0.6 ml/min PBSE (PBS/2 mM EDTA). Fractions of 0.6 ml were collected. Only fractions eluting at 7.4-9.2 ml (fraction A); 9.2-11.0 ml (fraction B) and 12.8-15.2 ml (fraction C) are collected. Fractions eluting at other volumes are stored at −20° C. until further use.
3. Measure protein concentrations using BCA reagent according to the manufacturer's instructions.
4. In order to obtain a good antigen mixture different amounts of fractions A, B and C were mixed, labelled and tested for different ELISA types. Mixed fractions A, B and C in: a 1:1:1 ratio (w/w) is a very good mixture for capture- and ICx-ELISA and is called RISA200.
   Labelling of Mycobacterium Tuberculosis (MTB) Antigen Mixtures
   Fluorescein-ULS Labelling of Antigens (All Mixtures e.g. KT3, KS98, RISA75 and RISA200)
1. Measure protein concentration with BCA-reagent (Pierce).
2. Dilute TB-antigens to a protein conc. of 0.6 mg/ml and add 200 μl Fluorescein-ULS (1 mg/ml in 10 mM NaOH, KREATECH LK 1004) per ml of protein.
3. Incubate 4 hrs at 50° C.
4^{a}. For the Capture-ELISA format Stabilzyme-Guard (Surmodics, SG660) is added (protein-concentration is 0.2 mg/ml) and the mixture is stored at 4° C.
4^{b}. For ICx-ELISA and indirekt coating ELISA, Flu-ULS labelled antigens are separated from unreacted Flu-ULS by gel filtration. PD10 columns (AP-Biotech, 17-0851-01) were used for this purpose. One ml was applied and eluted in 2.0 ml PBSE. The fluorescein concentration is determined spectroscopically by measuring fluorescein absorbance at 495 nm. Other ULS labels such as DNP-ULS and Biotin-ULS, were used in labelling of antigenic mixtures. All labels displayed comparable results. Also, other labelling methods, such as NHS labelling, were tested. For example, in fluorescein labelling of antigen mixtures a fluorescein-LC-NHS ester was used according to the manufacture's instructions (Molecular Probes, FF-4005).
   ELISA Methods for Serological Detection of MTB Infections
   Direct Coating ELISA
1. High-binding 96-well plates (Greiner, 12×8 strips) are coated overnight at RT with 100 μl of 4 μg/ml KT3 or KS98 in 0.1 M ammonium carbonate or 10 μg/ml RISA200 in PBS pH7.4.
2. The plates are washed 4 times with PBS/0.05% Tween80.
3. Plates are post-coated with 300 μl 3% BSA in PBS/0.05% Tween80.
4. Dilute serum 1:300 in dilution buffer A (DB-A: PBS/1% BSA/2% casein/0.05% Tween80/0.05% thimersal, 0.02% bromophenol blue). Pipet 100 μl/well and incubate 1 hr at 37° C.
5. The plates are washed 4 times with PBS/0.05% Tween80.
6. Pipet 100 μl Rb-anti-human IgG-HRP (Kreatech, □-chain specific), diluted in DB-A in each well and incubate 1 hr at 37° C.
7. The plates are washed 4 times with PBS/0.05% Tween80.
8. Detection is performed for 30 min at RT with 100 μl 0.04% TMB, the reaction is stopped with 100 μl 0.5 M sulphuric acid. Read absorbance at 450 nm with 0.620 nm as blanc.
   Capture ELISA
1. High-binding 96-well plates (Greiner, 12×8 strips) are coated overnight at RT with 200 μl of 4 μg/ml Rb-anti-human IgG (□-chain specific, DAKO, A0424).
2. The plates are washed 4 times with PBS.
3. Plates are post-coated with 300 μl 3% BSA in PBS.
4. Dilute serum 1:300 in dilution buffer B (DB-B: PBS/1% BSA/2% casein/0.05% thimerosal/0.02% phenol Red). Pipet 200 μl/well and incubate 1 hr at 37° C.
5. The plates are washed 4 times with PBS/0.1% BSA.
6. Pipet 0.200 μl of 2 μg/ml FLAME (Fluorescein Labelled Antigen MixturE, total protein concentration determined using BCA reagent and BSA as standard), diluted in DB-B in each well and incubate 1 hr at 37° C.
7. The plates are washed 4 times with PBS/0.1% BSA.
8. Pipet 200 μl Rb-anti fluorescein-HRP (Kreatech), diluted in DB-B in each well and incubate 1 hr at 37° C.
9. The plates are washed 4 times with PBS/0.1% BSA.
10. Detection is performed for 30 min. at RT with 200 μl 0.08% TMB, the reaction is stopped with 100 μl 0.5 M sulphuric acid. Read absorbance at 450 nm with 620 nm as blanc.
   Immunocomplex ELISA (ICx-ELISA)
1. High-binding 96-well plates (Greiner, 12×8 strips) are coated overnight at RT with 100 μl of 10 μg/ml Rb-anti fluorescein (DAKO, V4044)
2. The plates are washed 4 times with PBS.
3. Plates are post-coated with 300 μl 3% BSA in PBS.
4. Dilute serum 1:100 in dilution buffer B (DB-B). Add one volume of 250 ng/ml FLAME (fluorescein concentration as determined by absorbance at 495 nm), mix and immediately pipet 100 μl/well and incubate 1 hr at 37° C.
5. The plates are washed 4 times with PBS.
6. Pipet 100 μl Rb-anti-human IgG-HRP (Kreatech), diluted in DB-B in each well and incubate 1 hr at 37° C.
7. The plates are washed 4 times with PBS.
8. Detection is performed for 30 min. at RT with 100 μl 0.04% if TMB, the reaction is stopped with 100 μl 0.5 M sulphuric acid. Read absorbance at 450 nm with 620 nm as blanc.
   Indirect Coating ELISA
1. High-binding 96-well plates (Greiner, 12×8 strips) are coated overnight at RT with 100 μl 10 μg/ml Rb-anti fluorescein (DAKO, V4044).
2. The plates are washed 4 times with PBS.
3. Plates are post-coated with 300 μl 3% BSA in PBS.
4. Dilute FLAME to 125 ng/ml (fluorescein concentration as determined by absorbance at 495 nm) in dilution buffer B (DB-B). Pipet 100 μl/well and incubate 1 hr at 37° C.
5. The plates are washed 4 times with PBS.
6. Dilute serum 1:100 in dilution buffer B (DB-B), pipet 100 μl/well and incubate 1 hr at 37° C.
7. The plates are washed 4 times with PBS.
8. Pipet 100 μl Rb-anti-human IgG-HRP (Kreatech), diluted in DB-B in each well and incubate 1 hr at 37° C.
9. The plates are washed 4 times with PBS.
10. Detection is performed for 30 min. at RT with 100 μl 0.04% TMB, the reaction is stopped with 100 μl 0.5 M sulphuric acid. Read absorbance at 450 nm with 620 nm as blanc.
   Note

The ELISA formats can also be assembled by making use of labelled MTB antibodies instead of labelled MTB antigens. In order to demonstrate this principle the direct ELISA method was chosen as model test system. The procedure is as described above (direct coating ELISA) with some minor modifications such as: (i) in step 4 the serum containing the MTB antibodies was Flu-ULS labelled, according to standard ULS protein labelling conditions (e.g. section Labelling of Mycobacterium Tuberculosis (MTB) antigen mixtures), prior to adding it to the 96 well plate; and (ii) in step 6 anti-human IgG-HRP was replaced by anti Flu-HRP. The sera used in this experiment were defined TB positive sera (T code) as well as TB negative sera (BB code). The following results were obtained:

| Sera | Code | OD value at 450 nm |
|---|---|---|
| TB positive | T35 | 1.307 |
| | T39 | 2.875 |
| | T1006 | 2.086 |
| | T7JN | 0.779 |
| TB negative | BB101 | 0.321 |
| | BB102 | 0.356 |
| | BB103 | 0.400 |
| | BB104 | 0.352 |

Serum Panels
Control Sera

Four different sera are pipetted at positions 1A-D, 6A-D, 7E-H and 8E-H and served as controls to compare different 96-well plates for analyses. The first three sera are TB positive sera (PC1, PC2 & PC3). These sera were obtained from BioRes and contain a high, moderate and low titre of anti-TB antibodies, respectively. The negative control (NC) consists of a pool of sera from blood donors (obtained from CLB, Amsterdam, the Netherlands).

Venlo Serum Panel (VE/TB−)

Total Venlo serum panel contains 279 serum samples (VE001-VE279) from a peripheric hospital in Venlo (the Netherlands). These patients were tested positive in serological assays for diseases other than tuberculoses.

Healthy Donor Serum Panel (BB/TB)

Total healthy donor serum panel contains 555 sera (BB001-BB555) obtained from healthy blood donors at the CLB (Amsterdam, the Netherlands).

WHO TB Positive Serum Panel (WHO/TB+)

A total of 171 TB positive sera were obtained from the World Health Organization (WHO001-WHO171). All sera were found positive for TB using the standard culturing method. All sera were tested for HIV and found negative.

AIDS TB Positive Serum Panel (AIDS/TB+)

A total of 67 TB positive sera were obtained from the WHO(WHO50.1 WHO567). All sera were found positive for TB using the standard culturing method. All sera were tested positive for HIV infection.

160 Serum Panels (160A and 160 B)

The 160 serum panels always contain 60 WHO TB+ samples and 100 VE/TB-samples.

| | | |
|---|---|---|
| 160A | TB+ (WHO001-WHO060) no AIDS | TB− (VE001-VE100) |
| | patients | |
| 160B | TB+ (WHO101-WHO148) + 20% AIDS | TB− (VE101-VE200) |
| | patients (WHO501-WHO512) | |

Results and Discussion

Different purification methods were compared for their use in ELISA based methods for the detection of MTB antibodies in serum. Both, in culture supernatant secreted MTB antigens and cellular MTB materials were used as antigens. The latter were isolated by sonication and subsequent triton X100-extraction of MTB cells (KT3). Culture supernatant (glyco)proteins were ammonium sulphate precipitated and purified either by hydrophobic interaction chromatography (KS98) or by gel-filtration. For gel-filtration either Superdex75 (RISA75) or Superdex200 (RISA200) columns were used (FIG. 1).

The antigen fractions were used in different ELISA methods. These included: direct ELISA, indirect ELISA, capture ELISA, and in vitro immunocomplex (ICx) ELISA (FIG. 2). The individual fractions were analysed using serum panels consisting of 60 known MTB-positive samples (as determined by standard culture technique) and 0.100 MTB-negative samples. Positive samples were used to validate sensitivity and the negative to validate specificity of a given fraction. Results were presented in a ROC-curve format for each antigen fraction—test format combination. The greater the region under the curve the better the performance of the test. Fractions showing good sensitivity to specificity ratios were used for mixing. Also fractions with poor sensitivity and high specificity were sometimes used, however only when they possessed an additional value. An additional value of a fraction was that it detected MTB antibodies in sera that were negative for other fractions. Fractions with poor specificity or poor sensitivity without added value were not used. The protein content of each fraction was determined and fractions were mixed in different mass ratios and subjected in ELISA to a 160 serum panel (FIG. 3). The ratio with best ROC-curve was selected for further use. ELISA assays were used for the detection of both human IgG antibodies as well as—IgA antibodies. IgG detection resulted in better sensitivity because less than 50% of the IgG-positive samples were found positive with IgA detection. Less than 5% of the TB-positive samples were found positive in ELISA with anti-IgA detection alone. An additional effect of simultaneous detection of IgA and IgG antibodies was not found because the simultaneous detection also increased the number of false-positive samples in the sample collections (not shown).

FIG. 4 shows that the best results were obtained using Superdex200 purified fractions (RISA200 fractions), which were more specific than RISA75 fractions. This was due to the fact that some proteins with decreased specificity could be separated from desirable fractions. Gel filtration purified MTB secreted antigens were more sensitive than HIC purified antigen-mixtures (KS98). Also cellular material of MTB (KT3) can be used, however this is at the costs of specificity. Addition of even relatively small amounts of KT3 to secreted antigens to further optimise the antigen mixture decreased sensitivity/specificity.

For design of the ELISA formats, except for the direct ELISA, the antigen mixture was labelled with a hapten. Fluorescein was used because well defined antibodies against fluorescein can be used. Furthermore the high molecular absorbance of fluorescein simplifies determination of labelling performance. In order to label MTB antigens with fluorescein a cisplatin derivative coupled to fluorescein was used. This so called fluorescein-universal linkage system (FLU-ULS) forms a coordinative bond between the reactive platinum and aromatic N-donors (e.g. histidine) or S-donors such as methionines and free, non-sulphur bridged cysteines. FIG. 5 shows that results obtained with FLU-ULS are superior as compared to lysine- and N-terminus reactive fluorescein such as fluorescein N-hydroxy succinimide ester.

A preferred ELISA method was the in vitro immunocomplex method (ICx-ELISA, see FIG. 6). It is herewith shown that it is advantageous to use a method that does not bind the antigen mixture directly on to the surface of a (96-well) plate (direct ELISA). For example, this can be seen by the fact that the so-called indirect ELISA method that binds the antigen mixture on to the wells indirectly by means of an immunochemical reaction provided better sensitivity/specificity ratios. Both binding of patient-antibodies as well as detection of those antibodies with HRP labelled anti-human IgG or -IgA steps were comparable and the difference in performance might be due to the fact that coupling to the polystyrene wells is different or that the distance between antigen and solid surface is larger. Because the proteins are (partially) denatured before they can be absorbed to polystyrene this is likely to influence binding characteristics of MTB antibodies towards bound MTB antigens. Another ELISA approach tested was a capture approach. Polysterene wells were coated with anti-IgG or -IgA antibodies, followed by binding of patient IgG or -IgA which than bind fluorescein labelled MTB antigens. This approach is similar in performance as compared to indirect ELISA and gives better results as compared to direct ELISA. Specificity of the capture method was found better than indirect ELISA, however test sensitivity of the capture method was relatively low compared to indirect ELISA. This caused low positive patients to be tested negative and as a result, sensitivity of the tests decreased. Low positive patients have relatively low concentrations of MTB antibodies and because all IgG or IgA are captured before the antigen is bound, absolute amounts of captured MTB antibodies is low. The final approach combines the high specificity of a capture ELISA with the high sensitivity of indirect method. In ICx-ELISA FLU-ULS labelled antigens are immunochemically bound to the polystyrene well, however after being incubated with serum first. In principle, FLU-labelled immunocomplexes are formed between FLU-MTB antigens and MTB antibodies in solution. Subsequently, these complexes are captured by the coated anti-FLU antibodies. Immunoglobulin components of the bound immunocomplexes are visualised with HRP labelled anti-human IgG or -IgA antibodies. This method allows for the highest sensitivity/specificity ratios. Because the antigen is not in close contact with the solid support, and the reaction of antigen and antibody in solution are both beneficial for increased sensitivity as well as specificity as compared to the indirect ELISA. Both methods are essentially the same, however in indirect ELISA the immunocomplex is prepared step by step on carrier-bound components.

The FLU-ULS labelled RISA200-ABC1:1:1 mixture (ABC: antigen bar coding developed by Kreatech), combined with the ICx-ELISA method was used for analysis of a large sample panel. This panel consisted of all MTB positive and negative samples. At a specificity of 95% of all negative samples (n=834, false positive in ELISA n=41), 83% of the MTB positive/HIV negative samples (n=171, false negative in ELISA n=29) and 43% of the MTB positive/HIV positive samples (n=67, false negative in ELISA n=39) were found positive in ELISA. No significant difference in specificity was found between healthy donor- and sick-non TB samples.

### BRIEF DESCRIPTION OF THE DRAWINGS

FIG. 1: combined liquid chromatography profile of mycobacterium antigenic mixture RISA 75 and RISA 200 (absorption unit versus retention volume).

FIG. 2: Schematic representation of different ELISA methods.

FIG. 3: Effect of mixing A, B and C-fractions from RISA200 (ABC priciple). Determined by ICx-ELISA. Serum panel: 160B.

FIG. 4: Comparison of purification methods using capture-ELISA with serum panel 160A.

FIG. 5: Method of fluorescein binding (KS98) ULS vs NHS esters (capture ELISA, 160A panel.

FIG. 6: Comparison of different ELISA techniques using RISA200-ABC 1:1:1 as antigen (160B panel).

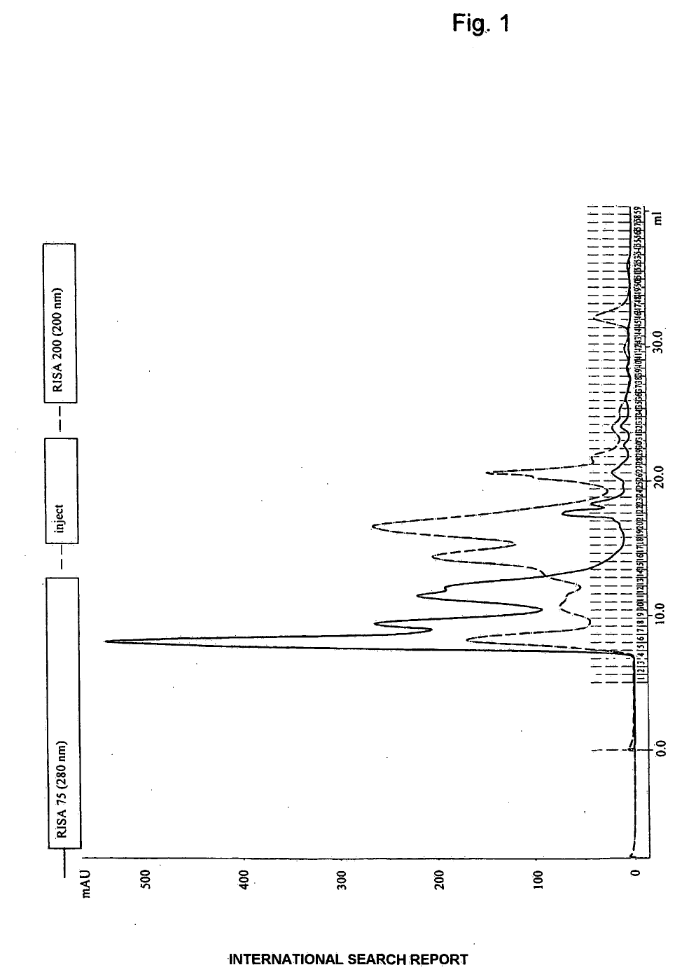

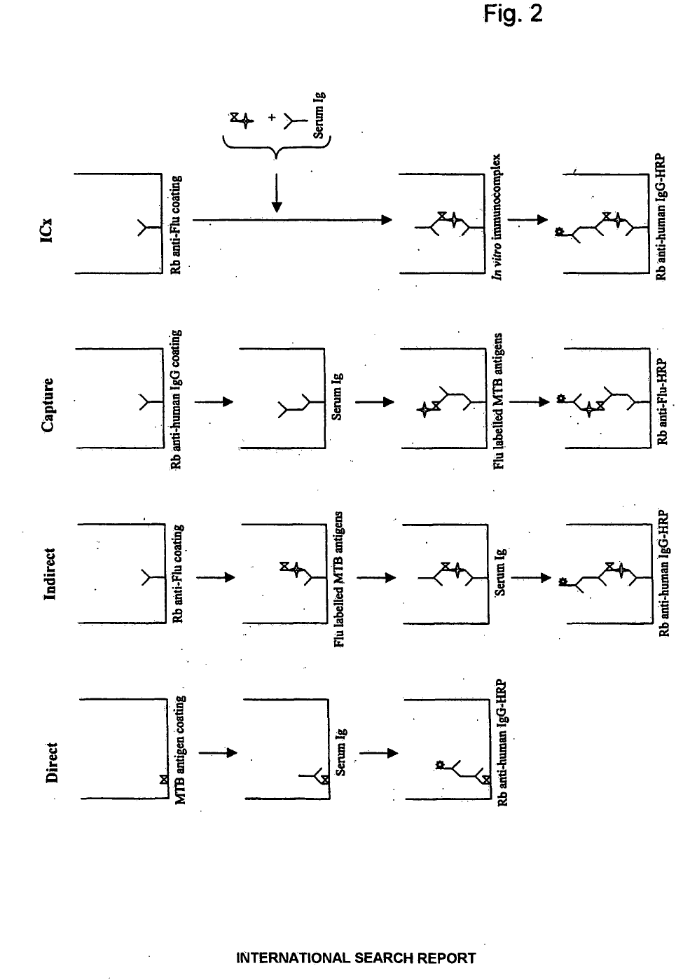

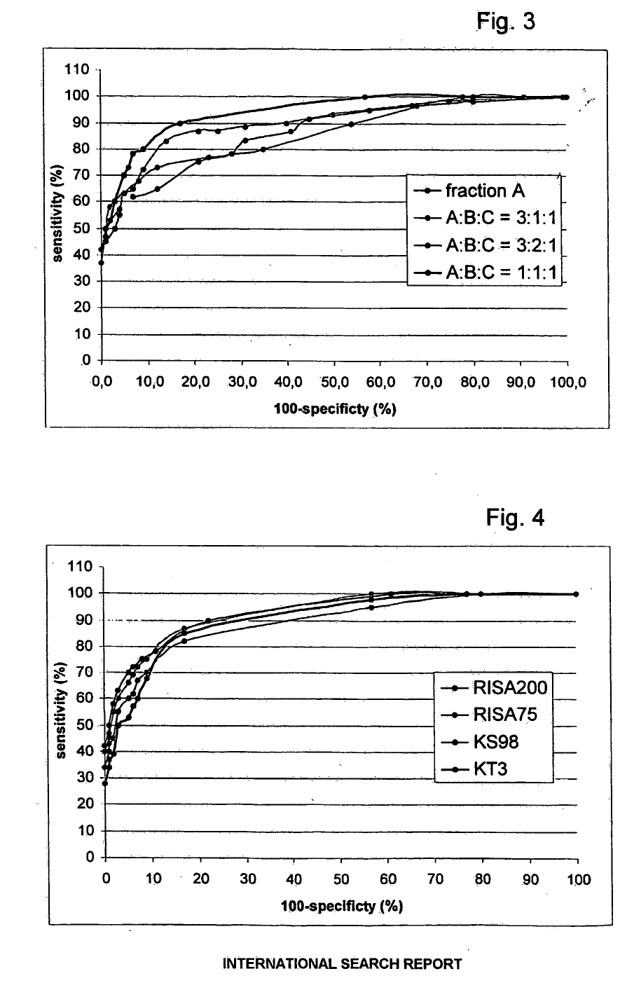

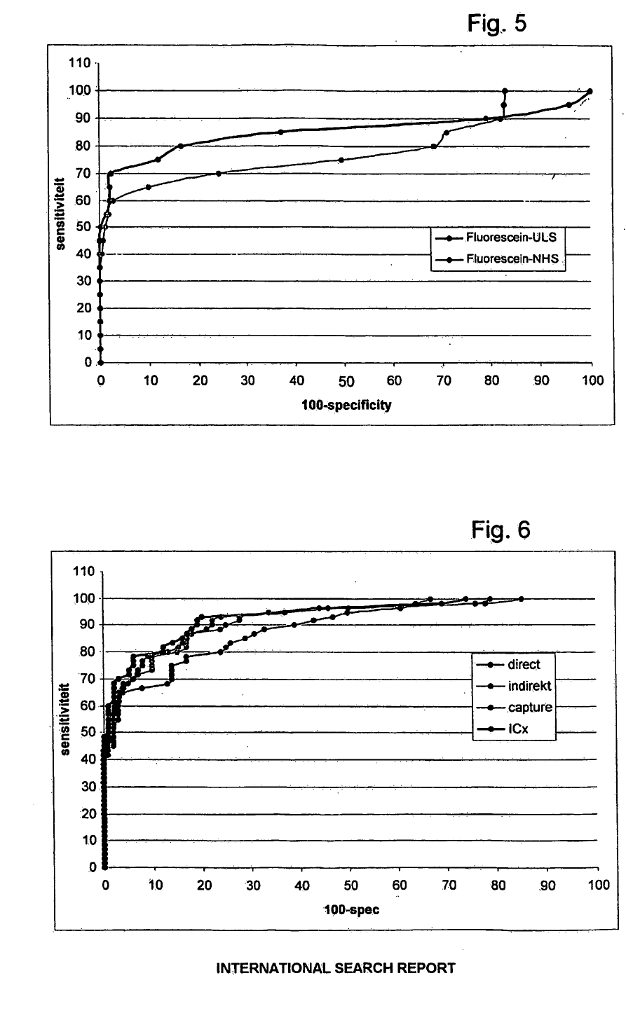

1. A method for determining whether an animal has been exposed to a mycobacterium or immunological equivalent thereof comprising providing a collection of mycobacterium antigens and determining whether a sample from said animal comprises an immunoglobulin capable of binding to at least one of said mycobacterium antigens, the method characterized in that binding of said antigen to a solid phase is achieved through a linking moiety.

2. A method according to claim 1, wherein binding of all of said antigens with a solid phase is achieved through a linking moiety.

3. A method according to claim 1, wherein at least one of said antigens further comprises a label.

4. A method according to claim 1, wherein said linking moiety comprises a label.

5. A method according to claim 3, wherein said label comprises a label for which a suitable capture reagents exists.

6. A method according to claim 3, wherein said label comprises fluorescein, dinitrophenol, biotin, digoxin, and/or digoxigenin.

7. A method according to claim 1, wherein said linking moiety comprises an immunoglobulin, or a functional equivalent thereof.

8. A method according to claim 7, wherein said immunoglobulin and/or said functional equivalent is specific for immunoglobulin of said animal.

9. A method according to claim 7, wherein said immunoglobulin and/or said functional equivalent is specific for at least one of said antigens.

10. A method according to claim 9, wherein said immunoglobulin specific for labelled antigen is specific for said label.

11. A method according to claim 1, wherein said antigens are bound to immunoglobulin prior to binding of said antigens to a solid phase, preferably via a linking moiety.

12. A method according to claim 11, wherein an immunoglobulin bound to said antigen prior to binding of said antigen to said solid phase comprises an immunoglobulin derived from said animal.

13. A method according to claim 1, wherein said collection comprises at least two different mycobacterium antigens.

14. A method according to claim 1, wherein at least one of said antigens is derived from the mycobacterium species for which said animal is being tested.

15. A method according to claim 1, wherein said collection comprises antigens purified from culture supernatant of cells comprising said mycobacterium.

16. A method according to claim 1, wherein said antigens are obtained by a method comprising purification of antigen by means of gelfiltration.

17. A method according to claim 1, wherein said binding of said antigen to a solid phase is achieved through an indirect ELISA, a capture ELISA and/or an immunocomplex ELISA.

18. A kit for performing a method according to claim 1, comprising a collection of mycobacterium antigens, wherein at least one of said antigens is bound to a solid phase through a linking moiety.

19. A kit according to claim 18, wherein all of said antigens are bound to a solid phase through a linking moiety.

20. A collection of mycobacterium antigens comprising at least one mycobacterium antigen that is physically linked to a label.

21. A collection according to claim 20, comprising a plurality of mycobacterium antigens physically linked to a label.

22. A collection according to claim 20, wherein said antigens are derived from supernatant from an in vitro culture of mycobacteria.

23. A collection according to claim 20, wherein said antigens have passed through a gelfiltration column.

24. A collection according to claim 20, wherein said label comprises fluorescein, dinitrophenol, biotin, digoxin, and/or digoxigenin.

25. A collection of antibodies comprising at least one antibody specific for a mycobacterium antigen physically linked to a label.

26. A collection according to claim 25, derived from the serum of an animal.

27. A collection according to claim 26, wherein said animal comprises a human.

28. A collection according to claim 25 wherein said label comprises fluorescein, dinitrophenol, biotin, digoxin, and/or digoxigenin.
